(19) United States
(12) Patent Application Publication
(10) Pub. No.: US 2025/0040989 A1
(43) Pub. Date: Feb. 6, 2025

(54) GUIDEWIRE AND CATHETER SELECTION AND REAL-TIME GUIDANCE

(71) Applicant: KONINKLIJKE PHILIPS N.V., EINDHOVEN (NL)

(72) Inventors: Rudolf Maria Jozef VONCKEN, EINDHOVEN (NL); Olaf VAN DER SLUIS, LANDGRAAF (NL); Paulus René Maria VAN BEERS, EINDHOVEN (NL); René Leonardus Jacobus Marie UBACHS, EINDHOVEN (NL)

(21) Appl. No.: 18/718,087

(22) PCT Filed: Dec. 13, 2022
(86) PCT No.: PCT/EP2022/085496

§ 371 Date: Jun. 10, 2024

Related U.S. Application Data
(60) Provisional application No. 63/290,392, filed on Dec. 16, 2021.

(51) Int. Cl. A61B 34/10; G16H 50/50

(52) U.S. Cl. CPC A61B 34/10; G16H 50/50; A61B 2034/104; A61B 2034/105; A61B 2034/107

(57) ABSTRACT

A vascular therapy apparatus (1) includes an electronic processing device (18) comprising at least one electronic processor (20) programmed to retrieve an intravascular instrument model (40) of an associated intravascular instrument (10) to be used in an intravascular procedure; retrieve a patient model (38) of anatomy including at least vasculature of a patient who is to undergo the intravascular procedure; and before and/or during the intravascular procedure, model a passage of the associated intravascular instrument through the vasculature of the associated patient using the instrument model and the patient model; and output treatment information for the intravascular therapy procedure based on the modeled passage of the associated intravascular instrument through the vascular of the associated patient.

### FIELD

The following relates generally to the intravascular procedure arts, catheter arts, thrombectomy arts, imaging arts, clot retrieval arts, simulation modelling arts, and related arts.

### BACKGROUND

For minimally invasive intravascular procedures, a large variety of instruments, such as guidewires and catheters, are available. In order to reach the treatment location, the surgeon makes an incision to access a blood vessel and inserts the distal end of the instrument into the blood vessel and then pushes the instrument through the vasculature until the tip reaches the treatment location. In doing so, the surgeon may need to navigate through blood vessel branch points, tortuous blood vessel sections, and other challenges. The instrument insertion may be done with medical imaging guidance, for example using an external X-ray or ultrasound imaging device and/or an intravascular ultrasound (IVUS) transducer mounted on the tip of the instrument, to provide limited visualization of the progress of the instrument through the vasculature. However, it is not always obvious which instrument would be the optimal one to use that can be guided to the target area in the least amount of time and with the least amount of damage to either tissue or device. Moreover, the medical imaging, even if available, can be difficult to interpret and may provide low or no contrast for important features such as vessel branch points. Successful insertion of the instrument to reach the treatment location can also depend on where the initial incision is made for entry of the instrument into the vasculature, but again the surgeon has limited guidance on where to make this incision.

During navigation of the instrument through tissue of a patient, there is a risk of tissue and device damage which is up to the physician to avoid. There is no feedback (e.g., visual, haptic, or sound signals) to provide warning if certain thresholds are crossed. Additionally, the resistance felt at the handle of a catheter or guidewire by the physician is the total sum of the local resistances along its entire length. It does not provide information on potential localized mechanical stress concentrations of the tissue of the patient.

Due to the limited maneuverability of the instrument in the tissue, and the complex shape of the vascular path the instrument must follow, accurately reaching the desired location in each patient is a difficult and possibly iterative process. Part of this is caused by large variation in anatomy of the patients and the large number of available medical instruments.

The following discloses certain improvements to overcome these problems and others.

### SUMMARY

In some embodiments disclosed herein, a vascular therapy apparatus includes an electronic processing device comprising at least one electronic processor programmed to retrieve an intravascular instrument model of an associated intravascular instrument to be used in an intravascular procedure; retrieve a patient model of anatomy including at least vasculature of a patient who is to undergo the intravascular procedure; and before and/or during the intravascular procedure, model a passage of the associated intravascular instrument through the vasculature of the associated patient using the instrument model and the patient model; and output treatment information for the intravascular therapy procedure based on the modeled passage of the associated intravascular instrument through the vascular of the associated patient.

In some embodiments disclosed herein, a vascular therapy includes retrieving an intravascular instrument model of an associated intravascular instrument to be used in an intravascular procedure; retrieving a patient model of anatomy including at least vasculature of a patient who is to undergo the intravascular procedure; and before and/or during the intravascular procedure, modeling a passage of the associated intravascular instrument through the vascular of the associated patient using the instrument model and the patient model; and outputting treatment information for the intravascular therapy procedure based on the modeled passage of the associated intravascular instrument through the vascular of the associated patient.

One advantage resides in providing assistance in determining an appropriate or optimal medical instrument to be used in an intravascular medical procedure.

Another advantage resides in using predictive computational simulations of medical instrument insertion and navigation to determine an optimal type of instrument to be used in an intravascular procedure.

Another advantage resides in using predictive computational simulations of a patient to determine an optimal access location for the medical instrument in an intravascular procedure.

Another advantage resides in determining stress levels of tissue of a patient during an intravascular procedure in real time and issuing a warning when the stress levels are approaching a critical value.

Another advantage resides in increased patient safety during a medical procedure.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates an intravascular therapy apparatus in accordance with the present disclosure.

FIG. 2 diagrammatically illustrates a method of performing an intravascular therapy method using the apparatus of FIG. 1.

FIG. 3 diagrammatically illustrates an example visual rendering generated by the apparatus of FIG. 1.

### DETAILED DESCRIPTION

The following relates to an approach for providing a surgeon with guidance when inserting a guide wire or catheter. Additionally or alternatively, guidance may be provided in selecting the instrument (i.e. guide wire or catheter).

Presently, the surgeon selects the instrument based on his knowledge and expertise, sometimes assisted by reference to a computed tomography (CT) medical image or other medical image of the patient. The instrument is selected for characteristics such as diameter, stiffness, and tip configuration. The surgeon inserts the instrument, often about 1 meter or so into the patient although more generally the total insertion distance depends on the incision point where the instrument enters and the location of the treatment and the intervening vascular path. In doing so, the surgeon is guided by feel of the resistance sensed at the handle by which the surgeon manipulates the instrument, possibly guided by medical images acquired using an external imaging system (e.g. ultrasound (US)) or internal imaging apparatus such as an optical shape fiber which enables reconstruction of a three-dimensional (3D) image of the vessel lumen. The foregoing feedback is limited, and if an error is made then the surgeon can inadvertently damage vascular or surrounding tissue, and/or can damage the instrument itself.

In some embodiments disclosed herein, a digital twin (i.e. simulation model) of the instrument is combined, and interacting, with a digital twin of the patient vasculature, to provide the surgeon with more information. The instrument model may model aspects such as instrument diameter, stiffness, tip configuration, and so forth. The patient model may model the 3D geometrical layout of the vasculature as well as tissue properties (elasticity, moduli, density, etc.). The patient model may be a generic patient model, or may be a patient-specific model generated, for example, by adapting a generic patient model to the specific vasculature of the specific patient based on deformable registration of the generic patient model to a CT or other medical image of the specific patient. In another example, the patient-specific model can be generated solely from the imaging data.

In preparatory embodiments, the two digital twins are used to model the interventional procedure ahead of it actually being performed. In this way, potential issues can be predicted, such as tortuous portions of the path that may present high risk of an issue, or situations where the target site cannot be reached from a prospective entrance site. The output of these preparatory simulations may include instrument selection, determination of the entrance site for inserting the instrument into the vasculature, and a “road map” which in some embodiments may be presented as a 3D rendering of the vasculature with the instrument path delineated and troublesome points along the path annotated.

In periprocedural embodiments, the two digital twins are used during the process of inserting the instrument into the patient. Advantageously, these embodiments can receive additional inputs such as the instrument length inserted thus far and the resistance being sensed at the handle (and/or, in variant embodiments, resistance or other feedback sensors placed at the tip or intervening points along the instrument). Using such feedback instrumentation, the modeling can be adapted in real time to more closely mimic the real-world situation present during the insertion. The output here may include warnings to reduce the insertion rate as the instrument tip approaches a tortuous portion of the path or other sensitive area, along with the graphical road map showing the actual position thus far of the instrument and the remaining path of the instrument. In some examples, the output can direct the user on the optimal handling of the instrument such that it follows the correct path upon insertion (e.g., the proper axial rotation of a bend catheter tip to access the correct branch or bifurcation).

In a variant periprocedural embodiment, if real-time imaging data are available (either external imaging (e.g., external US) or internal imaging (e.g., optical shape fiber), then this can serve as further input to the modeling performed using the two digital twins.

In any of the foregoing embodiments, the modeling can optionally perform “what-if” scenarios to, for example, provide pre-procedure recommendations of the best instrument to use for a particular procedure or to provide periprocedural recommendations as to which vascular path to follow at a vascular branching location.

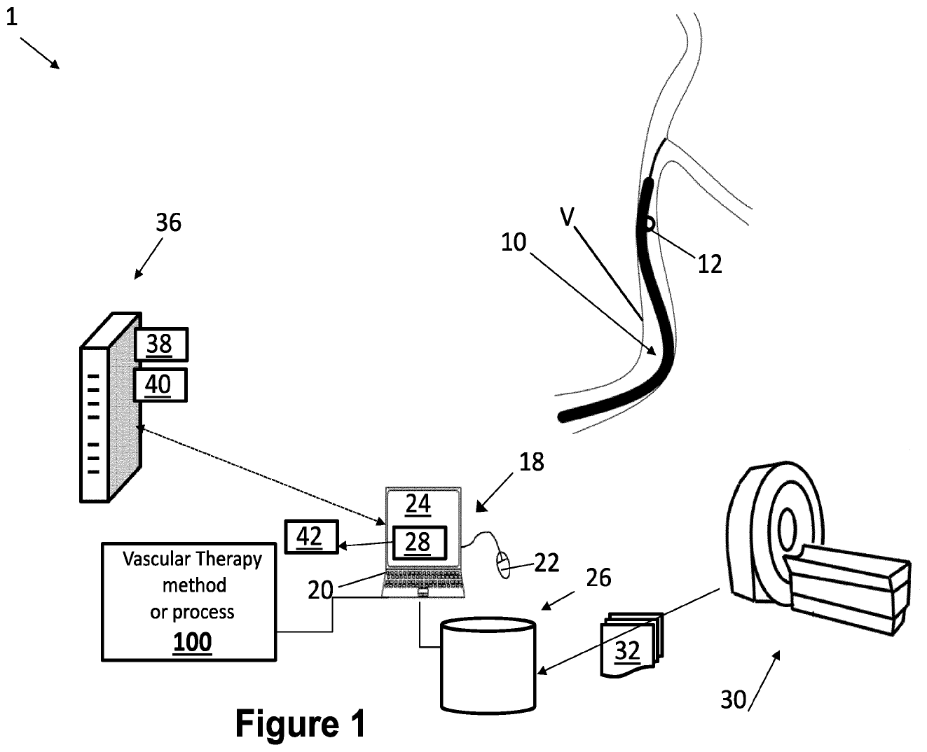

With reference to FIG. 1, an illustrative intravascular therapy (i.e., thrombectomy or atherectomy) apparatus 1 is diagrammatically shown. As shown in FIG. 1, the apparatus 1 includes an intravascular instrument 10 (e.g., a guidewire, a catheter, and so forth) for use in a vascular therapy medical procedure. In some examples, the medical instrument 10 can include one or more sensors 12 attached thereto or incorporated therein. The sensors 12 can measure location data of the medical instrument 10 when the intravascular instrument 10 travels through a blood vessel V of a patient undergoing the intravascular therapy procedure. The blood vessel V in general may be venous vasculature in the case of an intravenous therapy procedure (e.g., a thrombectomy), or may be arterial vasculature in the case of an intra-arterial therapy procedure (e.g., an atherectomy).

FIG. 1 further shows an electronic processing device 18, such as a workstation computer, or more generally a computer. The electronic processing device 18 may also include a server computer or a plurality of server computers, e.g., interconnected to form a server cluster, cloud computing resource, or so forth, to perform more complex computational tasks. The workstation 18 includes typical components, such as an electronic processor 20 (e.g., a microprocessor), at least one user input device (e.g., a mouse, a keyboard, a trackball, and/or the like) 22, and a display device 24 (e.g., an LCD display, plasma display, cathode ray tube display, and/or so forth). In some embodiments, the display device 24 can be a separate component from the workstation 18 or may include two or more display devices.

The electronic processor 20 is operatively connected with one or more non-transitory storage media 26. The non-transitory storage media 26 may, by way of non-limiting illustrative example, include one or more of a magnetic disk, RAID, or other magnetic storage medium; a solid-state drive, flash drive, electronically erasable read-only memory (EEROM) or other electronic memory; an optical disk or other optical storage; various combinations thereof; or so forth; and may be for example a network storage, an internal hard drive of the workstation 18, various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20. The instructions include instructions to generate a visualization of a graphical user interface (GUI) 28 for display on the display device 24.

FIG. 1 also shows an imaging device 30 configured to acquire images 32 of the intravascular instrument 10 during an intravascular procedure. The imaging device 30 is in communication with the at least one electronic processor 20 of the electronic processing device 18. In particular, in the illustrative examples the imaging device 30 comprises a CT imaging device 30; however, it will be appreciated that any suitable imaging device, such as, X-ray, ultrasound (US), magnetic resonance imaging (MRI), nuclear imaging, or any other suitable imaging device may be used. In some examples, the imaging device 30 can be a fluoroscopic imaging device (e.g., an X-ray imaging device, C-arm imaging device, a CT scanner, or so forth) for visualization of the intravascular instrument 10 in the blood vessel V of the patient.

The images 32 can be stored in the non-transitory storage media 26, or in a server computer 36. In addition, the server computer 36 can also store a patient digital twin model 38 (i.e., a simulation model) of the treatment site. The patient digital twin model 38 includes data related to a geometrical layout of vasculature of a patient, elasticity of tissue of the patient, moduli of tissue of the patient, and density of tissue of the patient. In one example, the patient digital twin model 38 can comprise a generic patient, while in another example, the patient digital twin model 38 is a patient-specific model adapted from a generic patient model with imaging data of a patient who is to undergo the interventional vascular therapy procedure.

The patient digital twin model 38 can be generated by using the acquired CT images 32, and the vasculature of the patient shown in the CT images 32 can be converted to a simulation grid on which a discretized form of relevant balance equations is solved (e.g., a finite element analysis, a finite volume analysis, a lattice Boltzmann analysis, a finite difference method analysis, and so forth). The motion and deformation of the intravascular instrument 10 are obtained from the physics-based calculations as a result of its dimensions, the (bio-) mechanical properties of the device (e.g., clastic stiffnesses of the parts assembled in the intravascular instrument 10) and surrounding patient tissue (e.g., anisotropic hyperelastic properties), tissue anatomy, interaction mechanisms between the device and the tissue (e.g., friction), and loading conditions of the device (e.g., prescribed force by the clinician). In addition, the patient digital twin model 38 can be updated through the constant inflow of new information from the images 32, the sensors 12, and so forth.

The server computer 36 also stores an intravascular instrument digital twin model 40 of one or more intravascular instruments (including the intravascular instrument 10). The intravascular instrument digital twin model 40 can include at least a diameter of the medical instrument 10, a stiffness of the medical instrument 10, a tip configuration of the medical instrument 10, and so forth.

The at least one electronic processor 20 is configured as described above to perform an intravascular therapy method or process 100. The non-transitory storage medium 26 stores instructions which are readable and executable by the at least one electronic processor 20 to perform disclosed operations including performing the intravascular therapy method or process 100. In some examples, the method 100 may be performed at least in part by cloud processing.

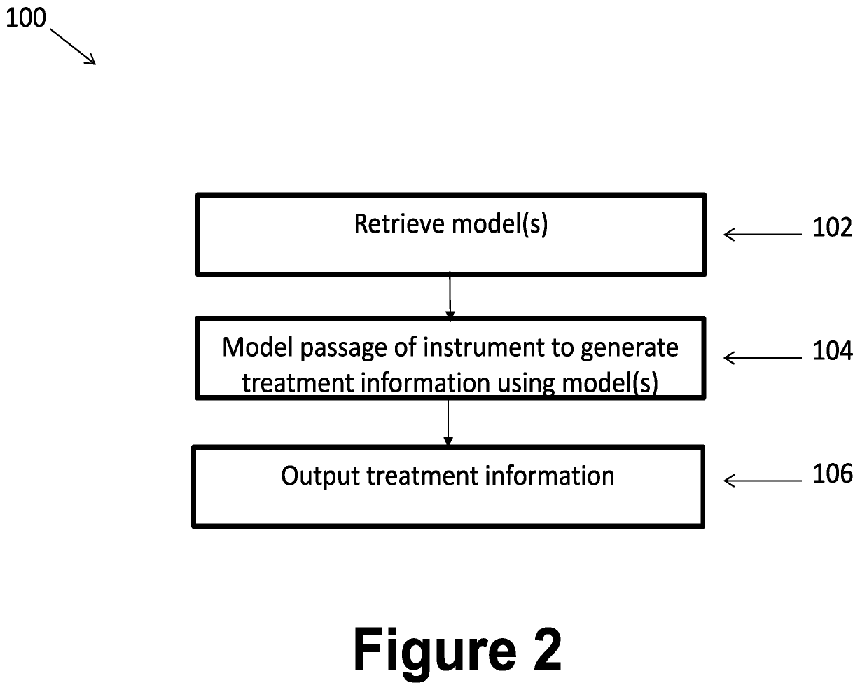

Referring to FIG. 2, and with continuing reference to FIG. 1, an illustrative embodiment of the vascular therapy method 100 is diagrammatically shown as a flowchart. At an operation 102, the patient digital twin model 38 and the intravascular instrument model 40 are retrieved from the server computer 36.

At an operation 104, which can be performed before and/or during an intravascular procedure using the medical instrument 10, a passage of the medical instrument 10 through the vascular (i.e., the blood vessel V) of the patient (or an average shape of the vasculature) can be modeled using the patient digital twin model 38 and the intravascular instrument model 40 to generate treatment information based on the modeling. This can be performed in a variety of manners.

In one example, the modeling operation 104 includes modelling the passage of the medical instrument 10 through the vasculature of the patient for a plurality of different candidate routes of the medical instrument 10 through the vasculature, and optionally also for a plurality of different candidate access sites (i.e., candidate incision sites for accessing the vasculature to initially insert the instrument). The treatment information includes a recommended route of the associated intravascular instrument through the vascular selected from the candidate routes selected based on the modeling, and optionally also a recommended access site. In the selection from amongst the different candidate routes, suitable metrics can be used to score each candidate route, such as total length of the route (with shorter routes being preferred), total number of vascular branches traversed along the route (with fewer traversed vascular branches being preferred), and/or a tortuousness metric measuring the number/sharpness of turns along the route (with straighter/less tortuous routes being preferred). In another example, the treatment information includes a recommendation to make a pre-bend on. for instance, a guidewire of the instrument 10. These are merely illustrative examples of some possible metrics for the route recommendation.

In another example, the modeling operation 104 includes modelling the passage of the medical instrument 10 through the vascular of the patient for a plurality of different candidate medical instruments 10 using different intravascular instrument models 40 corresponding to the different candidate intravascular instruments 10. The treatment information includes a recommended medical instrument 10 selected based on the modeling. In one approach for recommending the instrument, the vascular route can be analyzed to determine the turn with the smallest turn radius along the vascular route, a minimum blood vessel lumen diameter encountered through along the route, and a total length of the route. Any instrument whose minimum permissible turn radius is larger than the determined smallest turn radius along the route can then be excluded from recommendation. Similarly, any instrument whose diameter is larger than the minimum blood vessel lumen diameter along the route can be excluded from recommendation. Any instrument whose total insertable length is less than the total length of the route can be similarly excluded. These are merely illustrative examples of some possible metrics for the instrument recommendation.

In another example, the modeling operation 104 includes modelling a friction force on the medical instrument 10 as a function of position along the medical instrument 10 during the passage of the medical instrument 10 through the vascular. The treatment information includes identification of regions of high friction force on the medical instrument 10 during the passage of the medical instrument 10 through the vascular determined based on the modeling. Optionally, the quantitative friction force at each such region is also provided as treatment information.

In another example, the modeling operation 104 includes modelling tissue damage risk as a function of position along the vasculature during the passage of the medical instrument 10 through the vascular. The treatment information includes identification of vascular regions having high tissue damage risk determined based on the modeling. The tissue damage risk as a function of position can be modeled based on the corresponding friction force as a function of position and one or more tissue properties, such as vessel wall compliance, as a function of position.

In another example which is suitable in periprocedural embodiments, the at least one electronic processor 20 can receive sensor readings from the sensor(s) 12 during the intravascular procedure and perform the modeling operation 104 using the sensor readings along with the patient digital twin model 38 and the intravascular instrument model 40. In another example, the CT images 32, along with the patient digital twin model 38 and the intravascular instrument model 40, can be used to perform the modeling operation 104. In another example, the CT images 32 can be used to update the patient digital twin model 38 and/or the intravascular instrument model 40

In another example, the patient digital twin model 38 and the intravascular instrument model 40 can be used to perform what-if scenarios to generate the treatment information to be used in the interventional vascular therapy procedure. The what-if scenarios can include extrapolated data and current information about shape, position, insertion velocity, and so forth. In addition, other scenarios such as a change in insertion velocity (e.g. stopping or withdrawing), a rotation of the catheter or guidewire, change to another guidewire with another tip curvature and so forth can be simulated. The results of these simulations can be included in the treatment information. Such what-if scenarios can be run in preprocedural embodiments (for example, to model different candidate entrance points and different candidate routes, as previously described), and/or in periprocedural embodiments. In the latter case, the modeling can provide assistance, for example, when the surgeon encounters a vascular branching point, in order to provide guidance as to which vascular branch to follow. In another example, during a vascular therapy procedure, what-if scenarios can be performed and used as warnings of possible tissue damage not predicted prior to the vascular therapy procedure, but revealed by predictions of the model updated with information obtained during the ongoing vascular therapy procedure.

In other examples, which are suitable in periprocedural embodiments, the treatment information can also include one or more of a message to increase, decrease, or reverse an insertion speed of the medical instrument 10 into a patient, a graphic of a route of insertion of the medical instrument 10, and potential troublesome locations in the route of insertion of the medical instrument 10. In another example, the message can include recommendations pertaining to axial rotation, bending, and even the insertion speed of the medical instrument 10 by specifying whether the medical instrument 10 has to be advanced or retracted. These are all merely examples, and should not be construed as limiting.

At an operation 106, the generated treatment information is output, for example on the display device 24 of the electronic processing device 18. The outputting operation 106 can include generating and displaying a visual rendering 42 of the passage of the medical instrument 10 through the vascular with the regions of high friction force on the medical instrument 10 during the passage of the medical instrument 10 through the vascular indicated in the visual rendering 42. In another example, the visual rendering 42 can include the passage of the medical instrument 10 through the vascular with the vascular regions having high tissue damage risk indicated in the visual rendering. In another example, the visual rendering 42 can include additional data plotted next to or overlaid on one or more of the images 32 when displayed on the display device 24. The additional data can include, for example, a risk of damage levels to tissue and/or the medical instrument 10 based on a calculated mechanical loading on the tissue and the medical instrument 10. The simulations in this case can be based on pre-procedurally collected data of the patient geometry. These are all merely examples, and should not be construed as limiting.

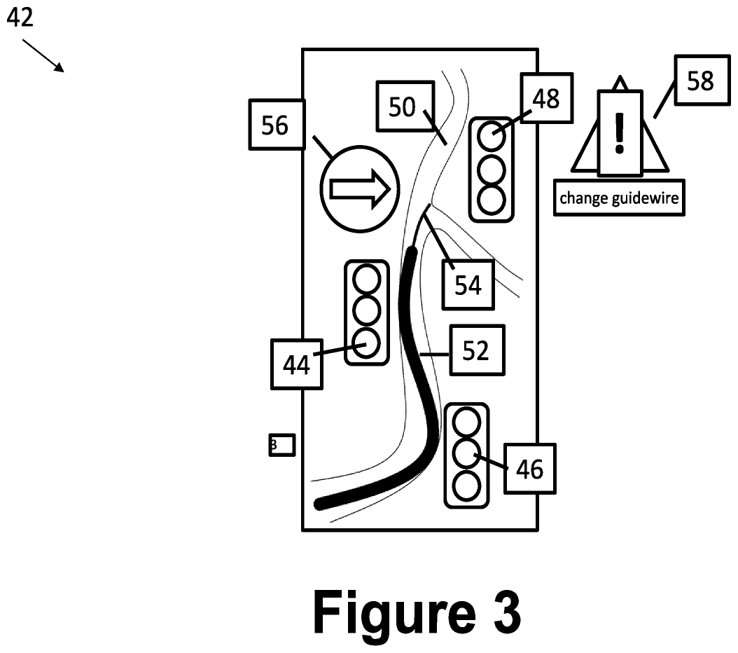

FIG. 3 shows an example of the visual rendering 42. The visual rendering 42 can include an indication of no or low risk of tissue damage (designated at 44), an indication of a medium risk of tissue damage (designated at 46), an indication of a high risk of tissue damage (designated at 48), the blood vessel (designated at 50), a representation of a catheter of the medical instrument 10 (designated at 52), a representation of a guidewire of the medical instrument 10 (designated at 54), an indication of a direction to move the medical instrument 10 (designated at 56), and an indication of a warning or advice (designated at 58).

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A vascular therapy apparatus, comprising:
an electronic processing device comprising at least one electronic processor programmed to:
retrieve an intravascular instrument model of an associated intravascular instrument to be used in an intravascular procedure;
retrieve a patient model of anatomy including at least vasculature of a patient who is to undergo the intravascular procedure; and
before and/or during the intravascular procedure, model a passage of the associated intravascular instrument through the vasculature of the associated patient using the instrument model and the patient model; and
output treatment information for the intravascular therapy procedure based on the modeled passage of the associated intravascular instrument through the vascular of the associated patient, wherein the modeling of the passage of the associated intravascular instrument through the vascular of the associated patient includes:
modeling friction force on the associated intravascular instrument as a function of position along the associated intravascular instrument during the passage of the associated intravascular instrument through the vascular.

2. The vascular therapy apparatus of claim 1, wherein the at least one electronic processor is programmed to:
model the passage of the associated intravascular instrument through the vascular of the associated patient for a plurality of different candidate routes of the associated intravascular instrument through the vascular,
wherein the treatment information includes a recommended route of the associated intravascular instrument through the vascular selected from the candidate routes selected based on the modeling.

3. The vascular therapy apparatus of claim 1, wherein the at least one electronic processor is programmed to:
model the passage of the associated intravascular instrument through the vascular of the associated patient for a plurality of different candidate associated intravascular instruments using different intravascular instrument models corresponding to the different candidate associated intravascular instruments,
wherein the treatment information includes a recommended associated intravascular instrument selected based on the modeling.

4. The vascular therapy apparatus of claim 1,
wherein the treatment information includes identification of regions of high friction force on the associated intravascular instrument during the passage of the associated intravascular instrument through the vascular determined based on the modeling.

5. The vascular therapy apparatus of claim 4, wherein the output of the treatment information includes:
generating a visual rendering of the passage of the associated intravascular instrument through the vascular with the regions of high friction force on the associated intravascular instrument during the passage of the associated intravascular instrument through the vascular indicated in the visual rendering.

6. The vascular therapy apparatus of claim 4, wherein the modeling is performed during the intravascular procedure, and the at least one electronic processor is further programmed to:
receive sensor readings from one or more sensors attached to the associated interventional instrument; and
perform the modeling using the instrument model and the patient model and further using the received sensor readings.

7. The vascular therapy apparatus of claim 1, wherein the modeling of the passage of the associated intravascular instrument through the vascular of the associated patient includes:
modeling tissue damage risk as a function of position along the vasculature during the passage of the associated intravascular instrument through the vascular,
wherein the treatment information includes identification of vascular regions having high tissue damage risk determined based on the modeling.

8. The vascular therapy apparatus of claim 7, wherein the output of the treatment information includes:
generating a visual rendering of the passage of the associated intravascular instrument through the vascular with the vascular regions having high tissue damage risk indicated in the visual rendering.

9. The vascular therapy apparatus of claim 1, wherein the modeling is performed during the intravascular procedure, and the at least one electronic processor is further programmed to:
receive medical images depicting passage of the associated intravascular instrument through the vasculature during the intravascular procedure; and
perform the modeling using the instrument model and the patient model and further using the received medical images.

10. The vascular therapy apparatus of claim 1, wherein the treatment information comprises one or more of: a message to increase, decrease or reverse an insertion speed of the associated intravascular instrument into a patient, a graphic of a route of insertion of the associated intravascular instrument, and potential troublesome locations in the route of insertion of the associated intravascular instrument.

11. The vascular therapy apparatus of claim 1, wherein the at least one electronic processor is programmed to:
use the instrument model and the patient model to perform what-if scenarios to generate treatment information to be used in the interventional vascular therapy procedure.

12. The vascular therapy apparatus of claim 1, wherein the instrument model includes at least a diameter of the associated intravascular instrument and a stiffness of the associated instrument, and a tip configuration of the associated instrument.

13. The vascular therapy apparatus of claim 1, wherein the patient model includes a geometrical layout of vasculature of a patient, elasticity of tissue of the patient, moduli of tissue of the patient, and density of tissue of the patient.

14. The vascular therapy apparatus of claim 13, wherein the patient model comprises one of:
a generic patient model; or
a patient-specific model adapted from imaging data of a patient who is to undergo the interventional vascular therapy procedure.

15. A vascular therapy method, comprising:
retrieving an intravascular instrument model of an associated intravascular instrument to be used in an intravascular procedure;
retrieving a patient model of anatomy including at least vasculature of a patient who is to undergo the intravascular procedure; and
before and/or during the intravascular procedure, modeling a passage of the associated intravascular instrument through the vascular of the associated patient using the instrument model and the patient model; and
outputting treatment information for the intravascular therapy procedure based on the modeled passage of the associated intravascular instrument through the vascular of the associated patient, wherein the modeling of the passage of the associated intravascular instrument through the vascular of the associated patient includes:
modeling tissue damage risk as a function of position along the vasculature during the passage of the associated intravascular instrument through the vascular.

16. The vascular therapy method of claim 15, further including:
modelling the passage of the associated intravascular instrument through the vascular of the associated patient for a plurality of different candidate routes of the associated intravascular instrument through the vascular,
wherein the treatment information includes a recommended route of the associated intravascular instrument through the vascular selected from the candidate routes selected based on the modeling.

17. The vascular therapy method of claim 15, further including:
modelling the passage of the associated intravascular instrument through the vascular of the associated patient for a plurality of different candidate associated intravascular instruments using different intravascular instrument models corresponding to the different candidate associated intravascular instruments,
wherein the treatment information includes a recommended associated intravascular instrument selected based on the modeling.

18. The vascular therapy method of claim 15, further including:
receiving sensor readings from one or more sensors attached to the associated interventional instrument; and
performing the modeling using the instrument model and the patient model and further using the received sensor readings.

19. The vascular therapy method of claim 15,
wherein the treatment information includes identification of vascular regions having high tissue damage risk determined based on the modeling.

20. The vascular therapy method of claim 15, wherein further including:
receiving medical images depicting passage of the associated intravascular instrument through the vasculature during the intravascular procedure; and
performing the modeling using the instrument model and the patient model and further using the received medical images.
